(19) United States
(12) Patent Application Publication
(10) Pub. No.: US 2014/0236117 A1
(43) Pub. Date: Aug. 21, 2014

(54) Disposable Patient Interface

(71) Applicant: PULSE NEEDLEFREE SYSTEMS, INC., Lenexa, KS (US)

(72) Inventor: MICHAEL DUTCHER, Olathe, KS (US)

(73) Assignee: Pulse Needlefree Systems, Inc., Lenexa, KS (US)

(21) Appl. No.: 13/771,392

(22) Filed: Feb. 20, 2013

(51) Int. Cl. A61M 5/30

(52) U.S. Cl. CPC A61M 5/3007
USPC 604/500; 604/68

(57) ABSTRACT

The present invention is directed to a patient interface for use with an injector assembly, the patient interface having a removable cap, wherein removal of the cap after an injection disables the patient interface to prevent re-use.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

The administration of vaccines and medications has historically been accomplished by using syringes and needles. However, use of syringes and needles poses the risk of disease transmission among injection recipients and other injuries to patients and health care workers. Human blood borne diseases that may be transmitted through syringes and needles include Hepatitis B, Hepatitis C, and Human Immunodeficiency Virus. Animal blood borne diseases that may be transmitted through syringes and needles include porcine reproductive and respiratory syndrome, anaplasmosis and enzootic bovine leukosis. When disposed of inappropriately, used syringes and needles pose risks of sickness and injury to the community. Also, syringes and needles have practical limitations in their ability to disperse the injectate into tissue and may require special skills or techniques to deliver the injectate into certain depths of tissue, such as the intradermal layer.

Alternative methods of injecting liquid medications have been developed. One of the methods is use of needle-free injectors or jet injection systems. Jet injectors were developed as early as the 1940's. These injectors were multi-dose high-workload devices that drew vaccines from multi-dose liquid containers. Needle-free injectors delivered accurate injections at high speed and were successful during smallpox, measles, and influenza immunization campaigns. But despite obvious advantages, old models did not have any safety features that protected multi-use device components from becoming contaminated, thereby putting patients at risk of cross-contamination.

There have been attempts to overcome the problem of cross contamination by offering needle-free injection systems with disposable plastic jet-injection syringes or disposable needle-free cartridges. While these disposable plastic jet-injection cartridges may reduce the risk of cross contamination, they introduce several other problems such as complexity of operation, high manufacturing costs and slow injection administration processes. The high manufacturing costs, in particular, prevent disposable plastic jet-injection cartridges from being implemented in developing countries where needle-free technology is needed to prevent ancillary spread of disease in large scale immunization programs. These high manufacturing costs and slow processing times also preclude use in veterinary vaccination programs where cost, speed and simplicity of operation are important considerations.

There have also been attempts to overcome the problem of cross contamination by applying a single-use “protector cap” to a multi-use needle-free jet injector, where the protector cap was designed to shield contaminants from the multi-use components of the injector. However, existing single-use protector caps have not been entirely satisfactory.

### BRIEF SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present invention is directed to a patent interface for use in connection with an injector assembly. In an exemplary embodiment, the patient interface comprises an injection stream enclosure having first and second ends and defining a cavity, wherein the first end defines an injection stream enclosure opening; a cap removably affixed to the first end of the injection stream enclosure, wherein the cap defines a cap opening; and wherein the injection stream enclosure opening and the cap opening are aligned to define an open pathway through the patient interface. The patient interface further comprises a tissue base plate affixed to the second end of the injection stream enclosure, wherein the tissue base plate defines a tissue base plate opening, and wherein the tissue base plate opening is aligned with the injection stream enclosure opening and the cap opening. The patient interface also includes an internal partition affixed to the interior of the injection stream enclosure, wherein the internal partition defines an internal partition opening, and wherein the internal partition opening is aligned with the injection stream enclosure opening and the cap opening

The present invention is also directed to an injector assembly employing the patient interface. In an exemplary embodiment, the injector assembly comprises an injection nozzle for expelling an injectate; a removable patient interface; and a retention lock movable between a first unlocked position and a second locked position; wherein the patient interface is held in place over the injection nozzle by the retention lock when the retention lock is in the second locked position. The patient interface further comprises a cap, wherein the cap is held in place over the injection nozzle by the retention lock.

In an exemplary embodiment, the patient interface comprises an injection stream enclosure, wherein the injection stream enclosure and cap are configured to define a groove around the patient interface, and wherein the retention lock engages the groove to hold the patient interface in place.

In an exemplary embodiment, the injector assembly further comprises an injection interlock interface reversibly movable between a first injection preventing configuration and a second injection permitting configuration. When the patient interface is held in place by the retention lock, the injection interlock interface is maintained in the second injection permitting configuration and when the patient interface is not held in place by the retention lock, the injection interlock interface is maintained in the first injection preventing configuration.

In an exemplary embodiment, the injector assembly further comprises an ejector configured to return the injection interlock interface from the second injection permitting configuration to the first injection preventing configuration. The injection interlock interface is configured to eject the injection stream enclosure, but not the cap, when the injection interlock interface is partially returned to the first injection preventing configuration, and the injection interlock interface is configured to eject the cap when the injection interlock interface is returned to the first injection preventing configuration.

The present invention is also directed to a method for using the patient interface in connection with an injector assembly.

### BRIEF DESCRIPTION OF THE DRAWINGS

The present invention is described in detail below with reference to the attached drawing figures, wherein:

FIG. 1 is a perspective view of one embodiment of a patient interface of the present invention.

FIG. 2 is a cross-sectional view of the patient interface shown in FIG. 1.

FIG. 3 is an exploded cross-sectional view of the patient interface of FIG. 1 in connection with a patient and an injector assembly;

FIG. 4 is a cross-sectional view of the patient interface of FIG. 1 prior to installation on an injector assembly in accordance with one embodiment of the present invention;

FIG. 5 is a cross-sectional view of the patient interface of FIG. 1 installed on the injector assembly of FIG. 4.

FIG. 6 is a cross-sectional view of the patient interface of FIG. 1 in connection with the injector assembly of FIG. 4 after a first ejection stage.

FIG. 7 is a cross-sectional view of the patient interface of FIG. 1 in connection with the injector assembly of FIG. 4 after a second ejection stage.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies.

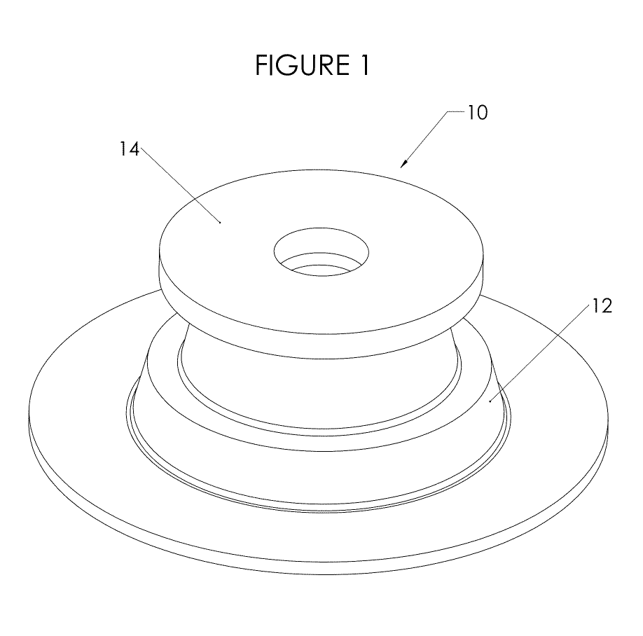

FIG. 1 depicts a patient interface 10 of one embodiment of the present invention, assembled and ready for use, showing injection stream enclosure 12 and cap 14 of patient interface 10.

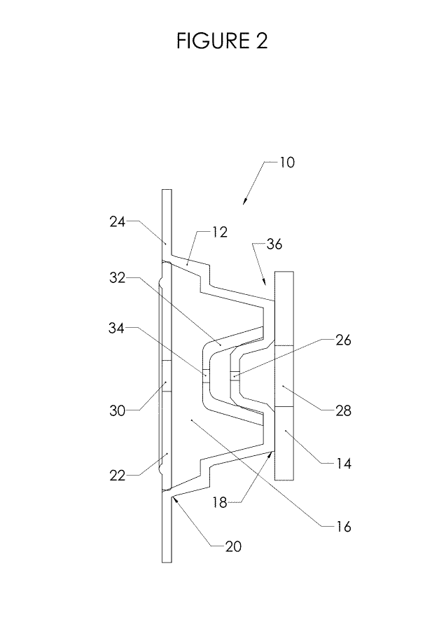

FIG. 2 depicts a cross sectional view of one embodiment of patient interface 10. Injection stream enclosure 12 is shaped to form cavity 16 within injection stream enclosure 12. Injection stream enclosure 12 has first end 18 and second end 20. Cap 14 is removably affixed to first end 18 of injection stream enclosure 12. Patient interface 10 further comprises tissue base plate 22 affixed to second end 20 of injection stream enclosure 12 to enclose cavity 16. Tissue base plate 22 may be formed separate from or integral with injection stream enclosure 12, or may not be present at all.

In the embodiment shown in FIG. 2, injection stream enclosure 12 comprises flange 24 around second end 20 of injection stream enclosure 12. Although flange 24 is shown encircling the lower edge of second end 20 of injection stream enclosure 12, flange 24 may be positioned around the exterior of injection stream enclosure 12 at a distance above the lower edge of second end 20 of injection stream enclosure 12 and may fully or partially encircle injection stream enclosure 12. In embodiments in which tissue base plate 22 is formed separate from injection stream enclosure 12, flange 24 alternatively may be formed integral with tissue base plate 22.

First end 18 of injection stream enclosure 12 defines an injection stream enclosure opening 26. Cap 14 defines cap opening 28. Tissue base plate 22 defines tissue base plate opening 30. Injection stream enclosure opening 26, cap opening 28 and tissue base plate opening 30 are aligned to define an open pathway through patient interface 10.

In the embodiment shown in FIG. 2, patient interface 10 further comprises an internal partition 32 within cavity 16, affixed to the interior of injection stream enclosure 12. Internal partition 32 may be formed separate from or integral with injection stream enclosure 12. Internal partition 32 defines internal partition opening 34, which is aligned with tissue base plate opening 30, injection stream enclosure opening 26 and cap opening 28 and further defines the pathway through patient interface 10.

In the embodiment shown in FIG. 2, the diameter of cap 14 is larger than the diameter of first end 18 of injection stream enclosure 12. Cap 14 and the exterior of injection stream enclosure 12 are formed to define a groove 36 between cap 14 and injection stream enclosure 12 around patient interface 10.

Injection stream enclosure 12, cap 14, tissue base plate 22 and internal partition 32 may be formed of any suitable material, as will be readily apparent to one of ordinary skill in the art. Injection stream enclosure 12, cap 14, tissue base plate 22 and internal partition 32, when formed separately, can be assembled in any manner known in the art, including friction fit, snap fit, screw fit, bayonet fit, heat-sealing or other means now known or hereinafter discovered to fit the components together into a functional unified interface in accordance with the present invention.

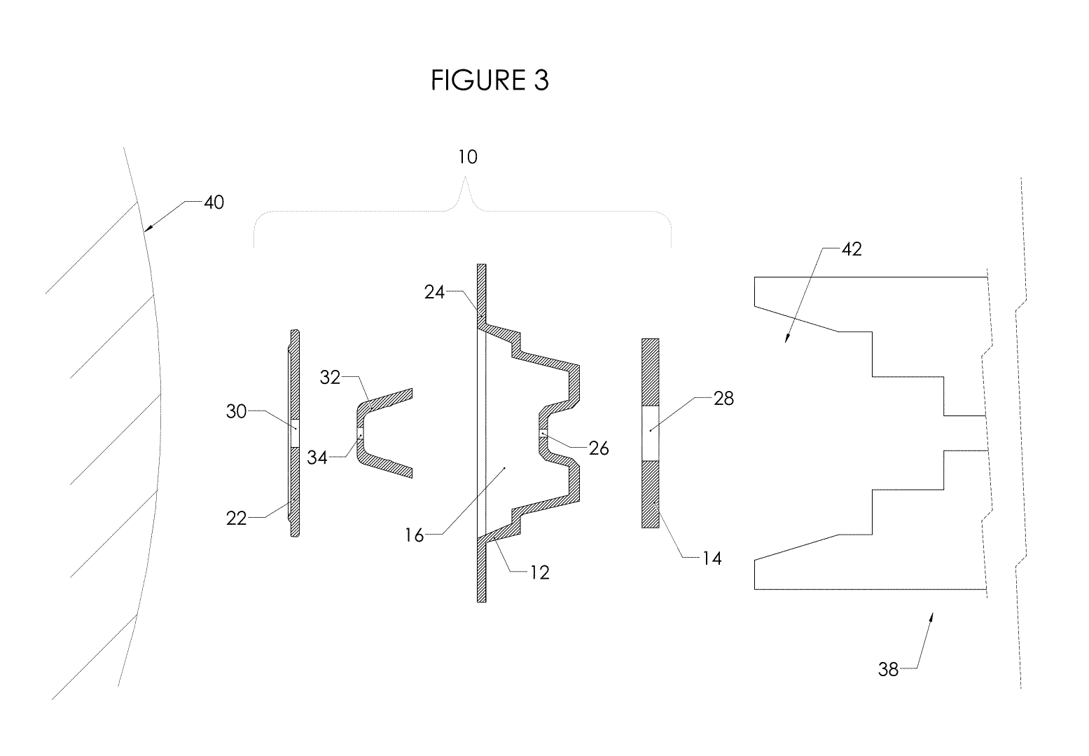

FIG. 3 depicts an exploded view of patient interface 10 with a simplified view of a needle-free injector assembly 38 and patient 40, in accordance with one embodiment of the present invention. One needle-free injector assembly suitable for use with the present invention is described in U.S. Pat. No. 7,887,506, incorporated herein by reference in its entirety. Injector assembly 38 has a patient interface cavity 42 at its distal end for receiving patient interface 10 within injector assembly 38.

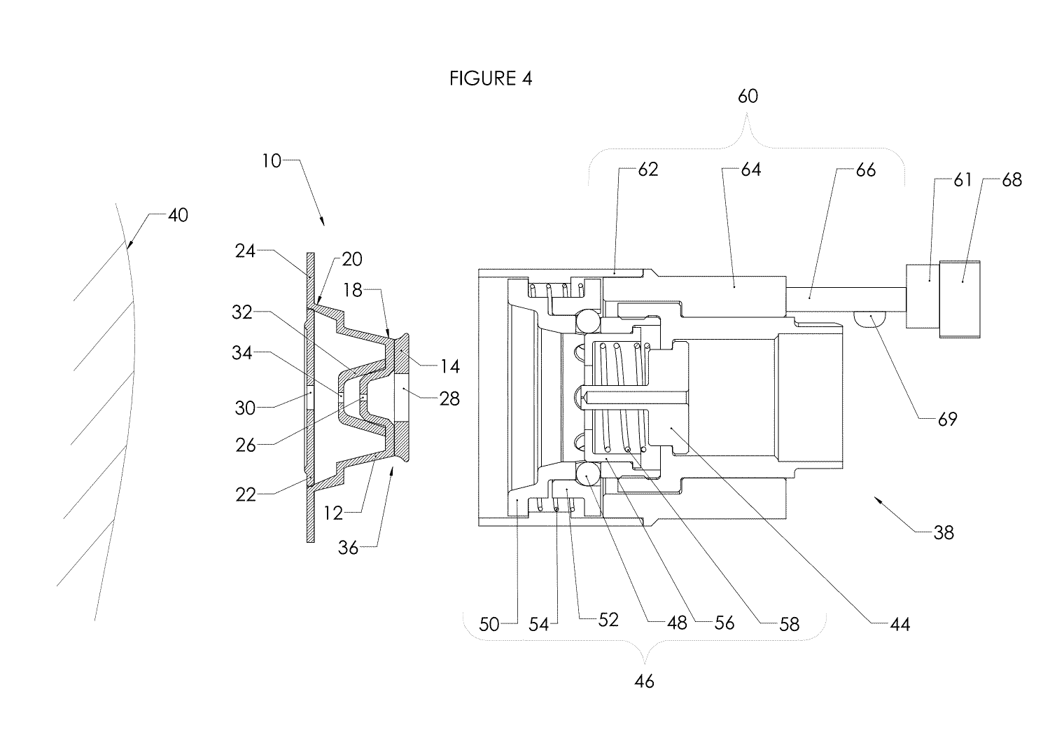

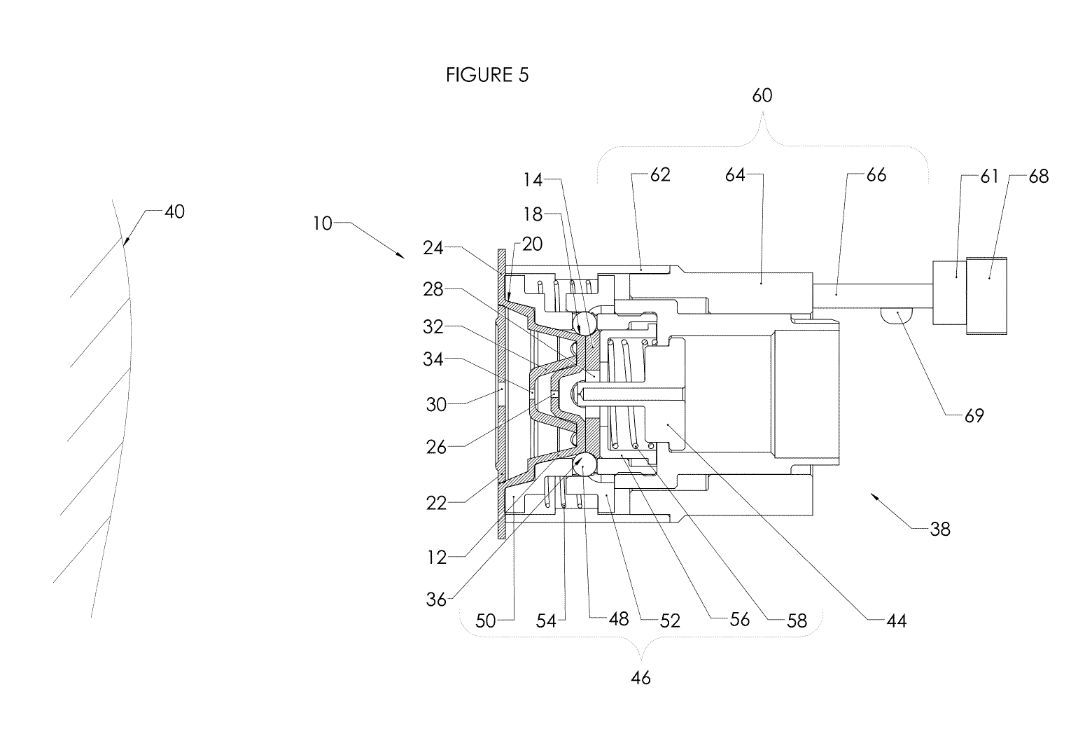

FIGS. 4-7 depict the patient interface 10 of FIGS. 1-3 in use in connection with an injector assembly 38 in accordance with one embodiment of the present invention. FIG. 4 depicts assembled patient interface 10 in its pre-injection state, ready to be installed on injector assembly 38 having an injection nozzle 44 for expelling an injectate. As shown in FIG. 5, patient interface 10 is received within patient interface cavity 42 and is positioned over the exterior of injection nozzle 44. Patient interface 10 is positioned with cap 14 positioned proximally to tissue base plate 22. A portion of patient interface 10, including at least a portion of cap 14, extends proximally into injector assembly 38 past the opening of injection nozzle 44.

Injector assembly 38 comprises retention lock 46 movable between a first unlocked position as shown in FIG. 4, and a second locked position as shown in FIG. 5. Retention lock 46 holds patient interface 10 in place over injection nozzle 44 when retention lock 46 is in its second locked position. In the embodiment shown in the Figures, the diameter of cap 14 is larger than the diameter of first end 18 of injection stream enclosure 12, and cap 14 is held in place by retention lock 46, which thereby holds patient interface 10 in place.

In the embodiment depicted in FIGS. 4-7, retention lock 46 is a ball-lock comprising a plurality of retention balls 48. As shown in FIG. 4, prior to installation of patient interface 10, when retention lock 46 is in its first unlocked position, retention balls 48 are held their unlocked position by stationary separator 50, retention collar 52 biased by retention collar spring 54 and insert 56 biased by insert spring 58.

Injector assembly 38 further comprises injection interlock interface 60, reversibly movable between a first injection preventing configuration as shown in FIG. 4 and a second injection permitting configuration as shown in FIG. 5. When patient interface 10 is not installed on injector assembly 38, injection interlock interface 60 is in its first injection preventing configuration, as shown in FIG. 4. When patient interface 10 is installed on injector assembly 38, patient interface 10 positions injection interlock interface 60 into the second injection permitting configuration, as shown in FIG. 5.

In the embodiment depicted in FIGS. 4-7, injection interlock interface 60 comprises a front pusher 62, rod pusher 64 and interlock rod 66. Front pusher 62, rod pusher 64 and interlock rod 66 may be formed separately or integrally as a single piece. Injection interlock interface 60 cooperates with the injection interlock 61 and injection interlock 61 cooperates with cocking mechanism 68 to prevent and allow injections. Injection interlock 61 may be a piston, latch, lock pin, pneumatic lock, magnet or other mechanism now known or hereafter developed that cooperates with cocking mechanism 68. Injection interlock interface 60 may be formed integrally with injection interlock 61.

Injection interlock interface 60 and injection interlock 61 are both reversibly movable between a first injection preventing configuration and a second injection permitting configuration. The injection preventing configuration is a configuration that prevents the expelling of any injectate through nozzle 44 or that prevents the expelling of an injectate at a pressure sufficient for injection into a patient. When injection interlock interface 60 and injection interlock 61 are in the first injection preventing configuration shown in FIG. 4, injection interlock 61 is not set to allow cocking mechanism 68 to initiate an injection. When the injection interlock interface 60 is in the injection permitting configuration, injection interlock 61 is set to allow cocking mechanism 68 to generate pressure sufficient for injection into a patient. An injection interlock and cocking mechanism consistent with the present invention are described in U.S. Pat. No. 7,887,506, incorporated herein by reference in its entirety, although any interlock and cocking mechanism now known in the art or later developed may be employed in injector assembly 38. Other cocking mechanisms, configurations of injection interlock interface 60 and injection interlock 61 and interactions between the components will be readily apparent to those of ordinary skill in the art.

FIG. 5 depicts a cross-sectional view of patient interface 10 installed on injector assembly 38 inside patient interface cavity 42. Tissue base plate 22 and flange 24 of injection stream enclosure 12 serve as the contact point between patient interface 10 and the patient 40. The pathway created by tissue base plate opening 30, internal partition opening 34, injection stream enclosure opening 26 and cap opening 28 is aligned with the opening of nozzle 44 of injector assembly 38 to create a pathway from nozzle 44 through patient interface 10 to the patient 40. Injection stream enclosure 12 serves to contain any materials emanating from the injection site when the injectate is injected into patient 40. Internal partition 32 creates an additional barrier to deflect materials away from injection stream enclosure opening 26, nozzle 44 and the remainder of injector assembly 38 during the injection. Injection stream enclosure 12 and internal partition 32 prevent contamination of nozzle 44 and the remainder of injector assembly 38.

In the embodiment of FIG. 5, retention lock 46 holds patient interface 10 in place on injector assembly 38 by cap 14. Retention balls 48 rest within groove 36 to hold cap 14 of patient interface 10 in place. Retention balls 48 are held in place in groove 36 by retention collar 52 biased by retention collar spring 54 and supported by separator 50. When patient interface 10 is held in place by retention lock 46, injection interlock interface 60 is maintained in its second injection permitting position, which resets injection interlock 61 to permit an injection.

One method for using patient interface 10 is described with respect to FIGS. 5-7. Turning first to FIG. 5, to install patient interface 10 on injector assembly 38, patient interface 10 is pushed proximally into patient interface cavity 42 of injector assembly 38. Cap 14 of patient interface 10 applies force against insert 56 to move insert 56 proximally further into injector assembly 38. Sufficient force must be applied against insert 56 to overcome the countervailing force of insert spring 58. The proximal movement of insert 56 and patient interface 10 aligns groove 36 of patient interface 10 with retention balls 48. The force provided by retention collar spring 54 causes retention collar 52 to apply force to retention balls 48, which are guided into groove 36 by separator 50. The force provided by retention collar spring 54 locks retention balls 48 in groove 36 of patient interface 10. Retention balls 48 hold patient interface 10 on injector assembly 38 by locking and holding cap 14 between insert 56 and retention balls 48.

Pushing patient interface 10 proximally into patient interface cavity 42 of injector assembly 38 also causes flange 24 of patient interface 10 to apply force against injection interlock interface 60 to move injection interlock interface 60 into its second injection permitting configuration. More specifically, in the embodiment of FIG. 5, flange 24 moves front pusher 62 proximally. The proximal movement of front pusher 62 moves rod pusher 64 proximally. The proximal movement of rod pusher 64 moves interlock rod 66 proximally. The proximal movement of the interlock rod 66 resets injection interface 61 to allow an injection.

Front pusher 62 also is configured to engage retention collar 52 when front pusher 62 is moved proximally. As described above, when patient interface 10 is in place in patient interface cavity 42, retention balls 48 are allowed to move into groove 36, thus allowing retention collar 52 to move proximally when engaged by retention collar 52. In one alternative embodiment, retention collar spring 54 is not present. In such embodiment, when patient interface 10 is pushed into patient interface cavity 42 and front pusher 62 is contacted by flange 24 of patient interface 10, front pusher 62 engages retention collar 52 and the proximal movement of front pusher 62, rather than retention collar spring 54, pushes retention collar 52 proximally.

If patient interface 10 is not in place, the pressure provided against retention balls 48 by insert 56 and insert spring 58 maintains retention balls 48 in their first unlocked position, blocking retention collar 52 from moving proximally. If retention collar 52 is unable to move proximally, it blocks proximal movement of front pusher 62. This would in turn prevent the further proximal movement of rod pusher 64 and interlock rod 66 required to reset injection interlock 61. Thus, before injection interlock 61 of the injector assembly 38 can be reset by injection interlock interface 60, a patient interface 10 must be placed into patient interface cavity 42 of injector assembly 38. If a patient interface 10 is not installed on the injector assembly 38, the proximal movement of front pusher 62 is restricted by retention collar 52 held in its first position by retention balls 48, and injection interlock interface 60 cannot be moved into its second injection permitting configuration.

With patient interface 10 in place in the patient interface cavity 42 and injection interlock 61 of injector assembly 38 reset to allow injection, tissue base plate 22 is placed in position with respect to patient 40 and an injection is administered to patient 40. For illustrative purposes, FIG. 5 depicts a separation of patient interface 10 from patient 40. However, in the preferred embodiment of the present invention, patient interface 10 is in direct contact with patient 40 during the injection. The injectate travels from nozzle 44 to patient 40 through the pathway defined by the aligned openings of patient interface 10, which are comprised of cap opening 28, injection stream enclosure opening 26, international partition opening 34 and tissue base plate opening 30. Once injection occurs, cocking mechanism 68 cannot initiate another injection until injection interlock 61 is reset to allow a subsequent injection.

After the injection is administered, patient interface 10 is expelled from injector assembly 38 by an ejector 69. Ejector 69 expels patient interface 10 by moving distally and thereby moving injection interlock interface 60 distally. Ejector 69 may be moved distally by a manual lever, manual drive, pneumatics, hydraulics, electromagnets or other mechanisms as will be readily apparent to one of ordinary skill in the art.

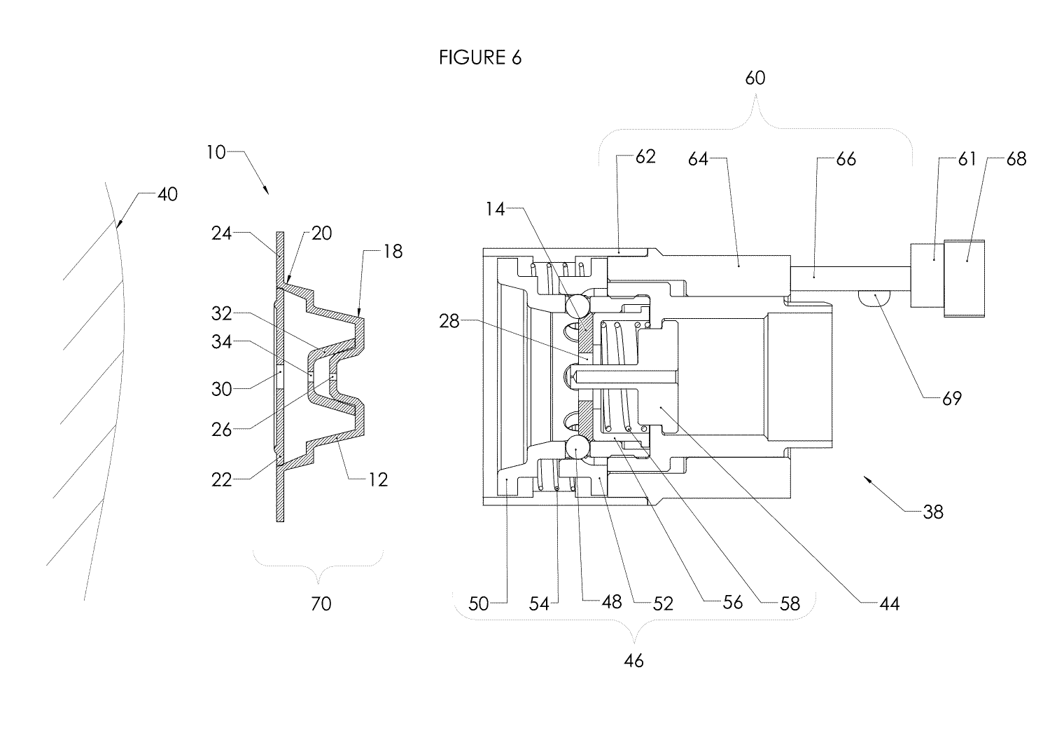

Patient interface 10 is ejected from injector assembly 38 in two stages. As shown in FIG. 6, in the first stage of ejection, injection stream enclosure 12 is separated from cap 14. When injection stream enclosure 12 is separated from cap 14, patient interface 10 is broken into at least two separate pieces. The first piece is cap 14. The second piece 70 is comprised of the remaining components of patient interface 10: injection stream enclosure 12, internal partition 32 and tissue base plate 22. Upon separation of patient interface 10 into two separate pieces, second piece 70 is no longer held in place on injector assembly 38 because cap 14 is the only portion of patient interface 10 held by retention lock 46. Second piece 70 of patient interface 10 free falls from injector assembly 38.

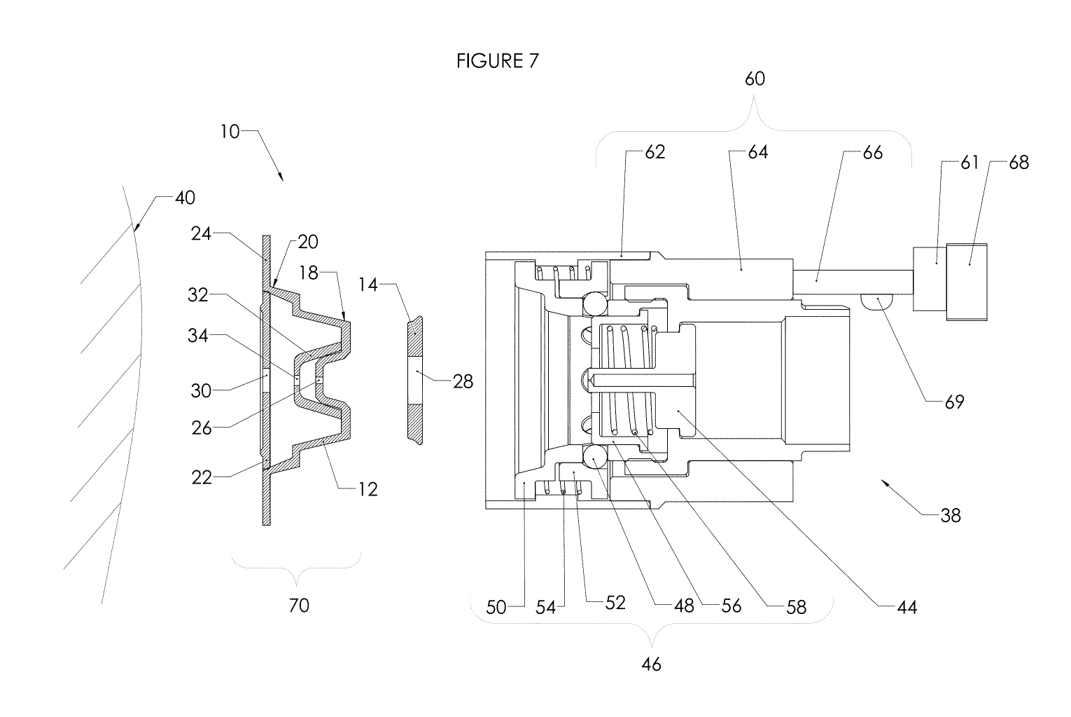

In the embodiment depicted in FIGS. 6 and 7, the first stage of ejection of patient interface 10 occurs when injection interlock interface 60 returns from its second injection permitting configuration, partially to its first injection preventing configuration. The first stage is initiated by ejector 69, which pushes interlock rod 66 distally. The distal movement of interlock rod 66 moves rod pusher 64 and front pusher 62 distally. In turn, the distal movement of front pusher 62 pushes against flange 24 to move second piece 70 of patient interface 10 distally. In the first stage of injection, cap 14 remains held against insert 56 by retention balls 48 of interface retention lock 46, while injection interlock interface 60 moves distally. Retention collar spring 54 stabilizes retention collar 52 against retention balls 48 during the first ejection stage. The distal movement of second piece 70 of patient interface 10, while cap 14 remains held in place by interface retention lock 46, creates a breaking point between cap 14 and injection stream enclosure 12 and separates the bond between these two components. With the bond between cap 14 and injection stream enclosure 12 broken, second piece 70 of patient interface 10 can free fall from injector assembly 38.

Turning to FIG. 7, after the free fall of second piece 70 of patient interface 10, cap 14 of patient interface 10 is released and ejected from injector assembly 38 in the second stage of the ejection process. In this stage, the distal movement of ejector 69 causes injection interlock interface 60 to fully return to its first injection preventing configuration. In the embodiment shown in FIG. 7, interlock rod 66, rod pusher 64 and front pusher 62 continue to move distally until rod pusher 64 engages the proximal side of retention collar 52. The force provided by rod pusher 64 is greater than the force of retention collar spring 54, and rod pusher 64 moves retention collar 52 distally to create a space around retention balls 48. This removes the force previously holding retention balls 48 in groove 36 of patient interface 10. The force of insert spring 58 moves insert 56 distally, which pushes cap 14 distally. This distal movement of cap 14 applies force to retention balls 48 and moves retention balls 48 from groove 36 to the space created by the distal movement of retention collar 52. The movement of retention balls 48 creates a pathway for cap 14 to be ejected from injector assembly 38. Insert spring 58 and insert 56 push cap 14 through this pathway to eject cap 14 from injector assembly 38.

Separation of second piece 70 and cap 14 of patient interface 10 physically disables patient interface 10 to prevent re-use of the used patient interface 10. The separation prevents a used patient interface 10 from being held on injector assembly 38 for a subsequent injection because cap 14 is necessary for retention of patient interface 10 on injector assembly 38. If patient interface 10 is not held in place by retention lock 46, injection interlock interface 60 is maintained in its first injection preventing configuration. If a user attempted to manually insert and hold one or both pieces of used patient interface 10 on injector assembly 38, it would be very difficult to provide sufficient pressure to overcome the force of insert spring 58 required to allow movement of injection interlock interface 60 into its second injection permitting configuration. Further, it would be very difficult to position patient interface 10 on injector assembly 38 to maintain alignment between cap opening 28 and the openings of second piece 70 of patient interface, as well as to maintain alignment between such openings and the opening of nozzle 44.

From the foregoing, it will be seen that this invention is one well adapted to attain all ends and objectives herein above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. A patient interface for use in connection with a separate injector assembly, comprising:
an injection stream enclosure having first and second ends and defining a cavity, wherein said first end defines an injection stream enclosure opening;
a cap removably affixed to said first end of said injection stream enclosure, wherein said cap defines a cap opening, and wherein said cap is not integrally connected to the injector assembly; and
wherein said injection stream enclosure opening and said cap opening are aligned to define an open pathway through the patient interface.

2. The patient interface of claim 1, further comprising a tissue base plate affixed to said second end of said injection stream enclosure, wherein said tissue base plate defines a tissue base plate opening, and wherein said tissue base plate opening is aligned with said injection stream enclosure opening and said cap opening.

3. The patient interface of claim 1, further comprising an internal partition affixed to the interior of said injection stream enclosure, wherein said internal partition defines an internal partition opening, and wherein said internal partition opening is aligned with said injection stream enclosure opening and said cap opening

4. The patient interface of claim 1, wherein said injection stream enclosure and said cap are configured to define a groove around said patient interface.

5. The patient interface of claim 4, wherein the diameter of said cap is greater than the diameter of said first end of said injection stream enclosure.

6. The patient interface of claim 1 further comprising a flange positioned around the exterior of said injection stream enclosure.

7. An injector assembly comprising:
an injection nozzle for expelling an injectate;
a removable patient interface; and
a retention lock movable between a first unlocked position and a second locked position;
wherein said patient interface is held in place over said injection nozzle by said retention lock when said retention lock is in said second locked position.

8. The injector assembly of claim 7, wherein said patient interface comprises a cap, and wherein said cap is held in place over said injection nozzle by said retention lock.

9. The injector assembly of claim 8, wherein said patient interface further comprises an injection stream enclosure, said injection stream enclosure and said cap are configured to define a groove around said patient interface, and said retention lock engages said groove to hold said patient interface in place.

10. The injector assembly of claim 9, wherein said retention lock is a ball lock.

11. The injector assembly of claim 9, wherein said cap defines a cap opening and said injection stream enclosure defines an injection stream enclosure opening, and wherein said cap opening and said injection stream enclosure opening are aligned with said nozzle to create a pathway from said nozzle through said patient interface.

12. The injector assembly of claim 9, wherein said injector assembly further comprises an injection interlock interface reversibly movable between a first injection preventing configuration and a second injection permitting configuration; and
wherein when said patient interface is held in place by said retention lock, said injection interlock interface is maintained in said second injection permitting configuration and when said patient interface is not held in place by said retention lock, said injection interlock interface is maintained in said first injection preventing configuration.

13. The injector assembly of claim 12, further comprising an ejector configured to return said injection interlock interface from said second injection permitting configuration to said first injection preventing configuration.

14. The injector assembly of claim 13, wherein said injection interlock interface is configured to eject said injection stream enclosure, but not said cap, when said injection interlock interface is partially returned to said first injection preventing configuration.

15. The injector assembly of claim 14, wherein said injection interlock interface is configured to eject said cap when said injection interlock interface is returned to said first injection preventing configuration.

16. The injector assembly of claim 7, wherein said injector assembly further comprises a patient interface cavity for receiving said patient interface.

17. A method for using a patient interface in connection with an injector assembly, comprising:
inserting a patient interface comprising an injection stream enclosure and a removable cap over a nozzle of an injector assembly, wherein said inserting step moves an injection interlock from a first injection preventing configuration to a second injection permitting configuration;
triggering said injector assembly to expel an injectate;
ejecting said injection stream enclosure from said injector assembly thereby separating said injection stream enclosure from said cap; and
ejecting said cap from said injector assembly after said step of ejecting said injection stream enclosure.
